(19) United States
(12) Patent Application Publication
(10) Pub. No.: US 2026/0033876 A1
(43) Pub. Date: Feb. 5, 2026

(54) SURGICAL SYSTEMS AND METHODS FOR BUNION REPAIR

(71) Applicant: Fusion Orthopedics USA, LLC, Mesa, AZ (US)

(72) Inventors: Adam J. Cundiff, Gilbert, AZ (US); Nathan G. Peterson, Gilbert, AZ (US); Eli W. Jacobson, Chandler, AZ (US)

(73) Assignee: Fusion Orthopedics USA, LLC, Mesa, AZ (US)

(21) Appl. No.: 18/788,949

(22) Filed: Jul. 30, 2024

(51) Int. Cl. A61B 17/80; A61B 17/17; A61B 17/72; A61B 17/86; A61B 17/90

(52) U.S. Cl. CPC A61B 17/8057; A61B 17/1728; A61B 17/725; A61B 17/7291; A61B 17/8061; A61B 17/808; A61B 17/8615; A61B 17/8625; A61B 17/90

(57) ABSTRACT

Surgical systems and methods for correcting a bunion through translating the capital fragment. The surgical system can include an implant including apertures. The surgical system can include a placement guide which can assist in placing the screws percutaneously through the implant and bone. The surgical system can include a rotation arm to facilitate achieving the desired amount of rotation for the capital fragment.

### FIELD OF THE TECHNOLOGY

The present technology relates generally to orthopedic surgery, and more particularly to, surgical instruments and systems for performing a minimally invasive bunion correction.

### BACKGROUND

It is desirable to have a minimally invasive approach to a bunion correction surgery because it minimizes trauma to patient tissues which in turn positively effects patient outcomes. Minimally invasive surgical approaches are difficult because visual access to the surgical site is limited or restricted. In many procedures radiographs are used to visualize steps of the surgical procedure and portions of the patient's anatomy. In a bunion correction, the placement of the bone screws requires a precision that is difficult to achieve with a radiograph. Thus, alignment tools would be helpful.

### SUMMARY

A surgical system can include an implant for bunion correction. The implant can include an attachment body and an extension. The attachment body and extension can include apertures of various characteristics. The surgical system can include a placement guide including an upper deck and a lower deck with aperture that can accept a drill guide. The placement guide can also include a handle. At least a portion of the bottom of the placement guide can be configured to conform to at least a portion of the top of the implant. The surgical system can include a rotation arm. The rotation arm can include a plurality of notches to hold a wire, which can facilitate rotating the capital fragment.

### BRIEF DESCRIPTION OF THE DRAWINGS

To readily understand the advantages and benefits of the technology, a more particular description of the technology briefly described above will be rendered by reference to specific embodiments that are illustrated in the appended drawings. Understanding that these drawings depict typical embodiments of the technology, and are therefore not to be considered to be limiting of its scope, the technology will be described and explained with additional specificity and detail through the use of the accompanying drawings, in which:

FIG. 1 is an isometric view of a schematic diagram illustrating an embodiment of a surgical system including an implant;

FIG. 2 is an end view of a schematic diagram illustrating the surgical system of FIG. 1 including a right and left implant;

FIG. 3 is a top view of a schematic diagram illustrating the surgical system of FIG. 2;

FIG. 4 is a bottom view of a schematic diagram illustrating the surgical system of FIG. 2;

FIGS. 5A through 5C are various views of a schematic diagram illustrating embodiments of a surgical system including a bone screw;

FIGS. 6 through 8 are various views of the implant of FIG. 1 and the bone screws of FIG. 5;

FIG. 9 is an isometric top view of a schematic diagram illustrating an embodiment of a surgical system including a placement guide;

FIG. 10 is an isometric bottom view of a schematic diagram illustrating the surgical system of FIG. 9;

FIG. 11 is a bottom view of a schematic diagram illustrating the surgical system of FIG. 9;

FIG. 12 is various views of a schematic diagram illustrating an embodiment of a surgical system including a drill guide and/or turret;

FIG. 13 is an isometric view of a schematic diagram illustrating an embodiment of a surgical system including a rotation arm;

FIG. 14 is a side view of a schematic diagram illustrating the surgical system of FIG. 13;

FIG. 15 is an isometric view of a schematic diagram illustrating the surgical system of FIG. 13 including a handle;

FIG. 16 is an isometric view of a schematic diagram illustrating the surgical system of FIGS. 1, 9, 12, and 13;

FIG. 17 is a side view of a schematic diagram illustrating the surgical system of FIG. 16;

FIG. 18 is an end view of a schematic diagram illustrating the surgical system of FIG. 16;

FIG. 19 is an isometric view of a schematic diagram illustrating the surgical system of FIG. 16;

FIG. 20 is an isometric view of a schematic diagram illustrating an osteotomy;

FIG. 21 is an isometric view of a schematic diagram illustrating a translated metatarsal capital fragment;

FIG. 22 is an isometric view of a schematic diagram illustrating the surgical system of FIG. 16 installed in the metatarsal of a subject;

FIG. 23 is an isometric view of a schematic diagram illustrating the surgical system of FIG. 22 including a rotated capital fragment;

FIG. 24 is an isometric view of a schematic diagram illustrating the surgical system of FIG. 23 with a k-wire;

FIG. 25 is an isometric view of a schematic diagram illustrating an embodiment of the surgical system of FIGS. 1, 9, and 12 on a subject;

FIG. 26 is an isometric view of a schematic diagram illustrating an embodiment of the surgical system of FIGS. 5A and 25 with one drill guide removed;

FIG. 27 is an isometric view of a schematic diagram illustrating an embodiment of the surgical system of FIG. 5A and 26 with the drill guide removed;

FIG. 28 is an isometric view of a schematic diagram illustrating an embodiment of the surgical system of FIG. 27 with the guide removed;

FIG. 29 is an isometric view of a schematic diagram illustrating an embodiment of the surgical system of FIG. 28 including a drill and drill guide;

FIG. 30 is an isometric view of a schematic diagram illustrating an embodiment of the surgical system of FIGS. 5B and 29 with the drill and drill guide removed;

FIG. 31 is an isometric view of a schematic diagram illustrating an embodiment of the surgical system of FIG. 30 including a drill over the k-wire;

FIG. 32 is an isometric view of a schematic diagram illustrating an embodiment of the surgical system of FIGS. 5C and 31; and

FIG. 33 is a flowchart illustrating an embodiment of a method of using the surgical system.

### DETAILED DESCRIPTION OF THE DRAWINGS

It should be understood that the language used in the present disclosure has been principally selected for readability and instructional purposes, and not to limit the scope of the subject matter disclosed herein in any manner. Further, reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including, but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. Aspects of the embodiments are described below with reference to flowchart diagrams and/or block diagrams of methods, apparatuses, and systems according to embodiments. The flowchart diagrams and/or block diagrams in the Figures illustrate the structure, functionality, and operation of possible implementations of apparatuses, systems, and methods according to various embodiments.

It should also be noted that, in some alternative implementations, the functions noted in the block may occur out of the order noted in the Figures. For example, two blocks shown in succession may, in fact, be executed substantially concurrently, or the blocks may sometimes be executed in the reverse order. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment.

The present technology may include any type of surgical system and is not limited to the style of surgical system depicted in the drawings. Furthermore, the described features, structures, or characteristics of the various embodiments may be combined in any suitable manner. One skilled in the relevant art will recognize, however, that embodiments may be practiced without one or more of the specific details, or with other methods, components, materials, and so forth. In other instances, well-known structures, and/or materials are not shown or described in detail to avoid obscuring aspects of an embodiment.

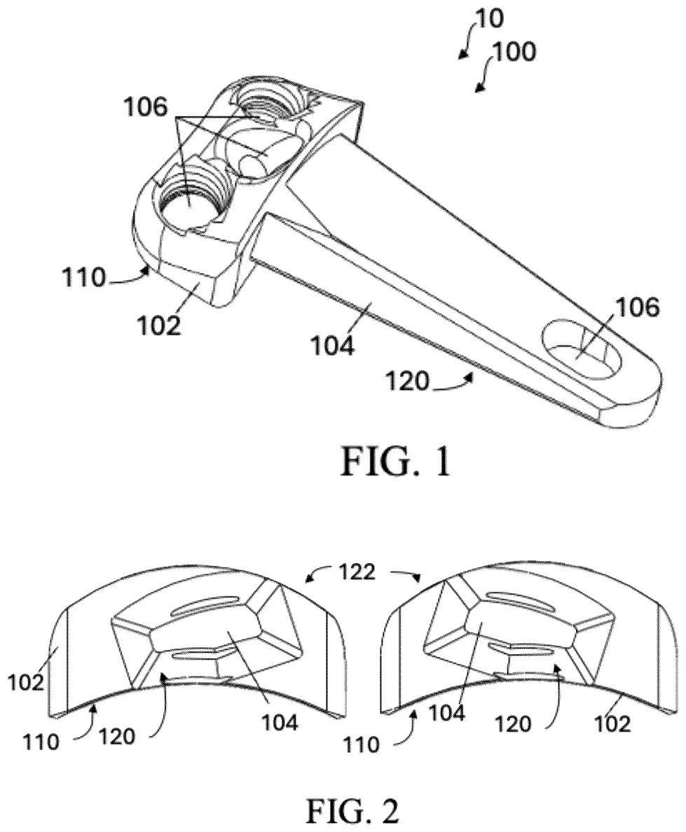

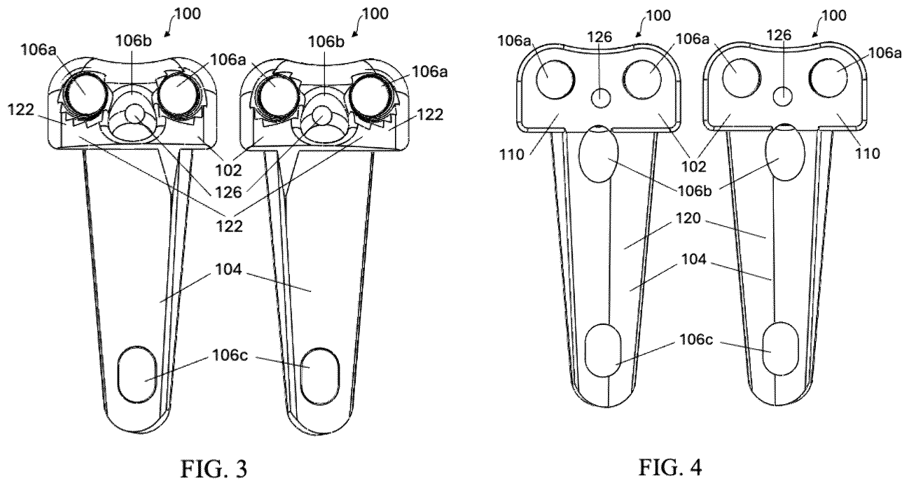

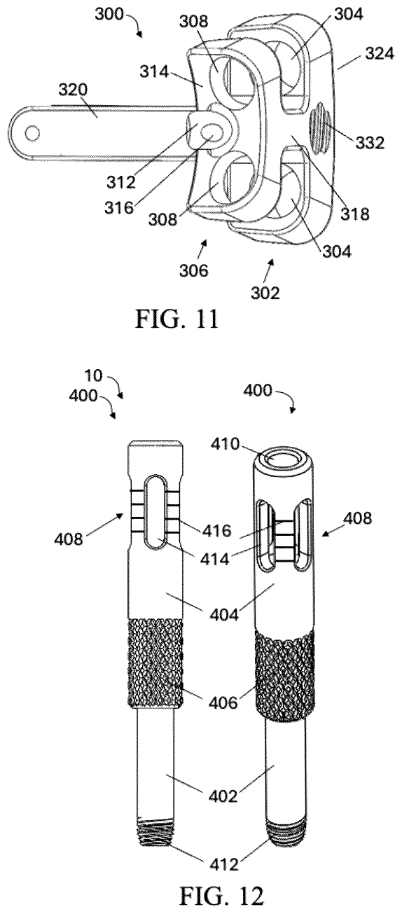

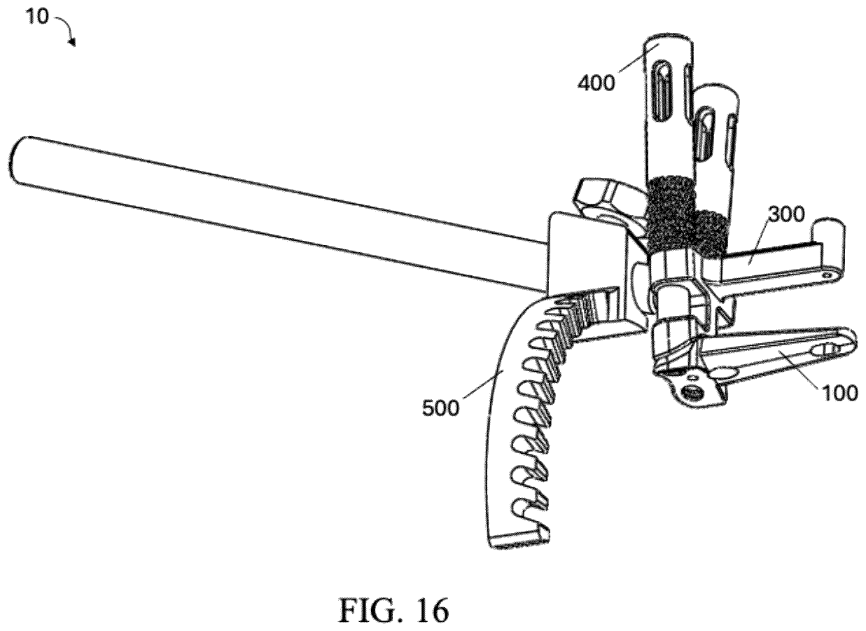

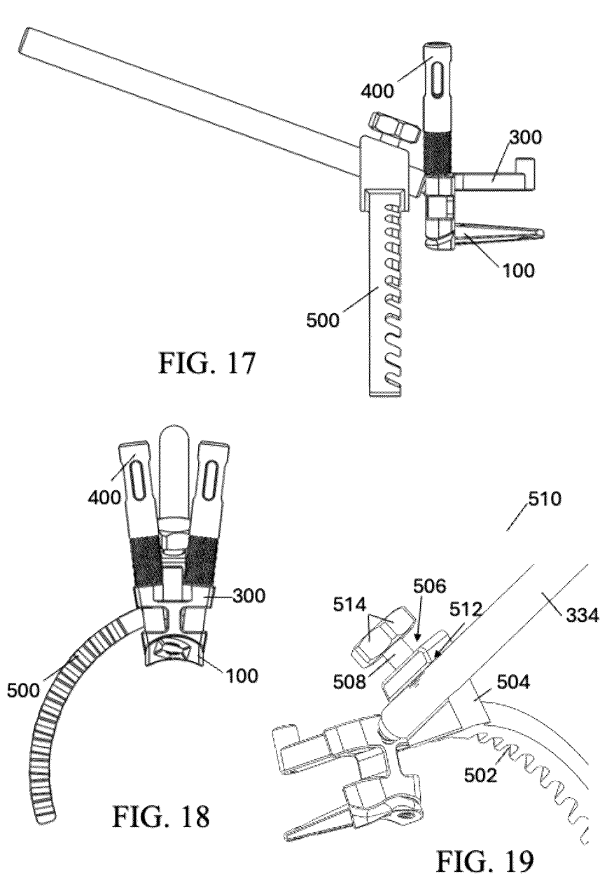

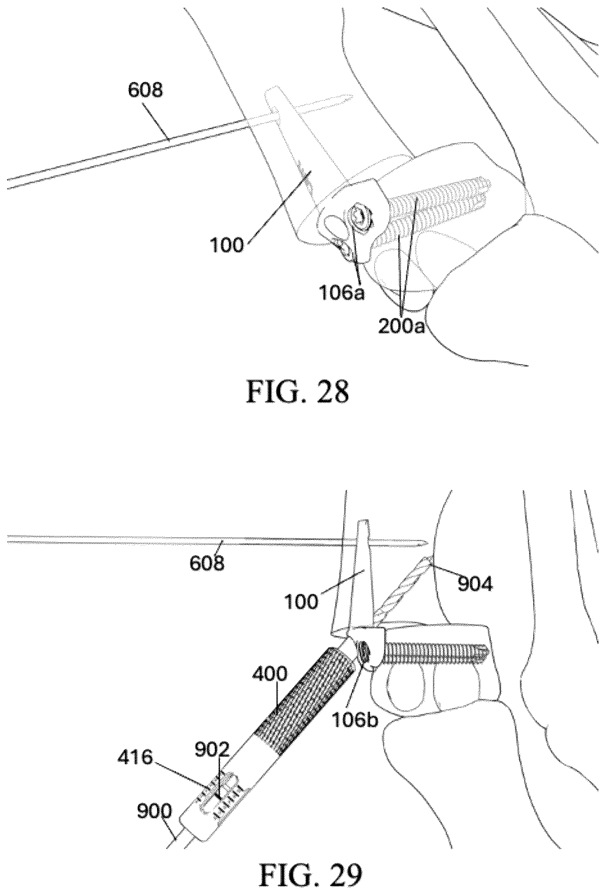

Turning now to the Figures, FIGS. 1 through 32 are diagrams illustrating various views and/or embodiments of a surgical system 10. In various embodiments, the surgical system 10 can be utilized to perform a bunion repair, and/or other procedures that are possible, each of which is contemplated herein.

The surgical system 10 may be constructed of any suitable material. In various embodiments, the surgical system 10 is constructed of a material that can be sterilized, and/or a material that is sterilized. In some embodiments, the surgical system 10 can include stainless steel, radio-opaque, radiolucent, titanium, titanium alloy, UHMW polyethylene, plastic, and/or aluminum, among other suitable materials that are possible, each of which is contemplated herein. In additional or alternative embodiments, the surgical system 10 includes surgical grade stainless steel, among other suitable surgical grade materials that are possible, each of which is contemplated herein. In some embodiments at least one portion of the system 10 can be sterilized.

Referring now to FIGS. 1 through 4. At least in the illustrated embodiment, the surgical system 10 includes, among other features, an implant 100. The implant 100 can include an attachment body 102. In some embodiments the attachment body 102 can include at least one aperture 106. The aperture 106 can be threaded, or non-threaded, oblong, countersunk, circular, oblong, fixed angle, adjustable angle, etc., among other features and/or characteristics, each of which is contemplated herein. In certain embodiments the attachment body 102 can include a plurality of apertures 106. In at least one embodiment each of the plurality of apertures 106 is the same. In other embodiments at least two apertures 106 are different.

In at least one embodiment, the implant can include an extension 104 including a longitudinal axis 108. In some embodiments, the extension 104 longitudinal axis 108 is the same as the implant 100 longitudinal axis 108. In certain embodiments, the extension 104 can include at least one aperture 106. In various embodiments the attachment body 102 can include at least two apertures 106a that are each threaded. In some embodiments the attachment body 102 can include at least a portion of aperture 106b that is non-threaded. In various embodiments the extension 104 can include at least a portion of aperture 106b. In certain embodiments, the extension 104 can include at least one aperture 106c. At least in the illustrated embodiment, the extension 104 can include at least one slot/elongated aperture 106c.

In one or more embodiments, the attachment body 102 can include a bottom 110 and the extension 104 can include a bottom 120. In various embodiments, the attachment body 102 bottom 110 can be configured to conform to a bone. In some embodiments the attachment body 102 bottom 110 can be configured to conform to a metatarsal capital fragment 604.

In one or more embodiments the implant 100 can be configured for more than one anatomical location, such as, for example, a left foot and a right foot. In other embodiments the implant 100 can be configured to conform to a specific anatomical location, such as, for example, a left metatarsal or a right metatarsal (see e.g., FIG. 2).

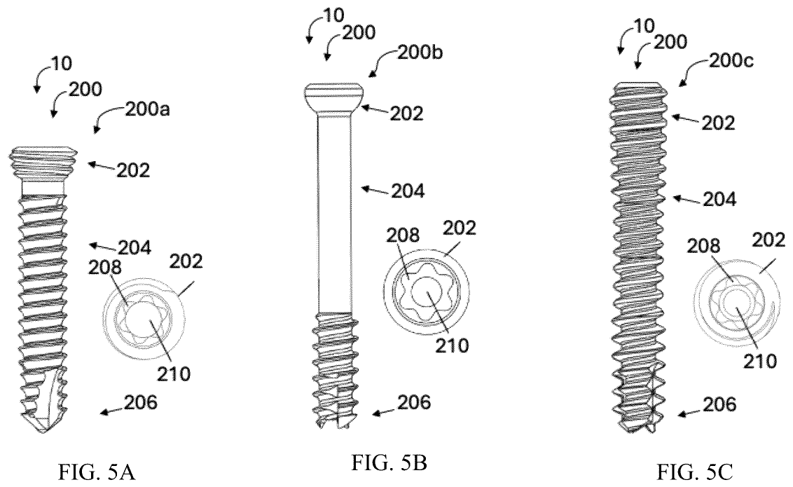

Referring now to FIGS. 5 through 8. In at least one embodiment, at least one aperture 106 is configured to accept a bone screw 200. The bone screw 200 can be at least one of a locking screw, a non-locking screw, a lag screw, a fully threaded screw, a nail, etc., among other bone screws that are possible, each of which is contemplated herein. In various embodiments the screw 200 can include an aperture 210 that extends through the length of the screw 200 (e.g., the screw can be cannulated). In one or more embodiments, the bone screw 200 can include a head 202, a shaft 204, and a tip 206. In certain embodiments the tip 206 can include a self-tapping and/or self-drilling contour and/or geometry. The self-tapping tip can assist in removing bone material which can eliminate the need for pre-drilling. The head 202 can include at least one of the following geometries: tapered, morse tapered, straight, threaded, non-threaded, locking, non-locking, etc., among other head geometries and/or characteristics that are possible, each of which is contemplated herein. In some embodiments the head can include an aperture 208 that can conform with a driving tool (e.g., a screwdriver) for driving the screw 200 into bone. The aperture 208 can be hexagon, torx, Phillips, flat, star, etc., among other shapes that are possible, each of which is contemplated herein. The shaft 204 can be at least one of partially threaded, fully threaded, smooth, etc., among other shaft characteristics that are possible, each of which is contemplated herein. In certain embodiments the surgical system 10 can include a plurality of bone screws 200. In some embodiments each of the plurality of bone screws 200 are a plurality of instances of the same screw. In other embodiments each of the plurality of bone screws 200 are different. In at least one embodiment the plurality of bone screws 200 is a mix of same and different screws. At least in the illustrated embodiment the surgical system 10 can include at least one locking screw 200a, at least one lag screw 200b, and at least one fully threaded screw 200c.

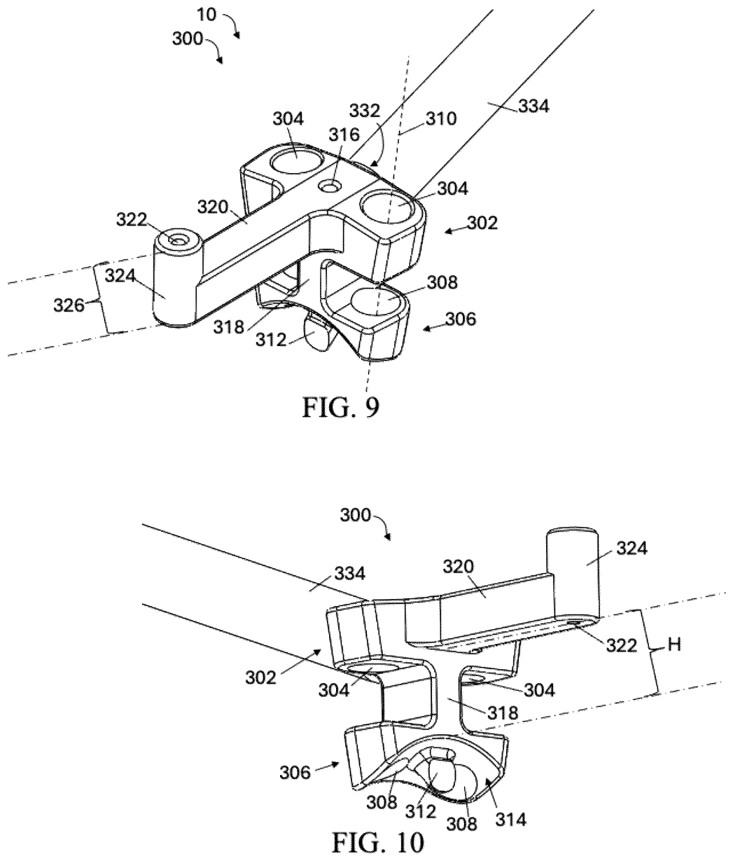

Referring now to FIGS. 9 and 10. In some embodiments, the surgical system 10 can include a targeting device and/or installation tool and/or placement guide and/or jig 300. In at least one embodiment, the guide 300 can include an upper deck 302. In at least the illustrated embodiment, the upper deck 302 can include at least one guide aperture 304. In some embodiments, the upper deck 302 can include a k-wire aperture 316. In certain embodiments, the guide 300 can include a lower deck 306. The lower deck 306, in one or more embodiments, can include at least one corresponding aperture 308 which can align with the guide aperture 304, such that a guide axis 310 can intersect the center of the guide aperture 304 and the center of the corresponding aperture 308. In many embodiments, the lower deck 306 can include a bottom surface 314. In some embodiments the bottom surface 314 can conform to the attachment body top surface 122 and can include an aperture plug 312. The aperture plug 312, at least in the illustrated embodiment, can include a protrusion configured to conform to at least one aperture 106. In some embodiments, the aperture plug 312 can include the k-wire aperture 316. At least in the illustrated embodiment the aperture plug 312 can be configured to conform to the aperture 106b. In certain embodiments, the upper deck 302 and the lower deck 306 are separated by a spacer 318 with a height of H. The spacer 318 height H can be in the range of five millimeters (5 mm) to twenty millimeters (20 mm) inclusive. In some embodiments the height H can be less than 5 mm. In other embodiments the height H can be greater than 20 mm.

In various embodiments, the guide 300 can include an extension 320. The extension 320 can include a k-wire guide 324. The k-wire guide 324 can be a body with a length 326 and a k-wire aperture 322.

In at least one embodiment, the guide 300 can include a handle 334 configured to couple with an aperture 332 on the guide 300. In some embodiments, the handle 334 can have opposing recessed rectangles 336 (see e.g., FIG. 27). In some embodiments, the aperture 322 can be generally orthogonal to the guide 300 such that the handle 334 is straight (see e.g., FIG. 25). In other embodiments, the aperture 322 can be angled relative to the guide 300 (see e.g., FIG. 9).

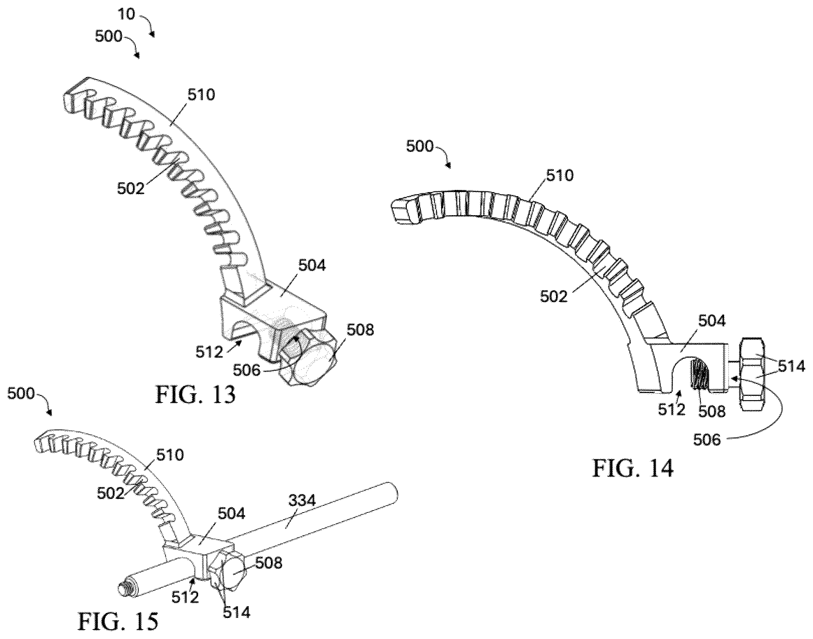

Referring now to FIG. 12. In one or more embodiments, the surgical system 10 can include at least one turret and/or a drill guide 400. The turret 400 can include a lower portion 402 and an upper portion 404. In some embodiments, the diameter of the lower portion 402 can smaller than the diameter of the aperture 304. In other embodiments the diameter of the lower portion 402 can be equal to the diameter of the aperture 304. The lower portion 402 can include threads 412. The threads 412 can be complimentary to threads in aperture 106 and/or aperture 106a. In some embodiments, the diameter of the upper portion 404 can be greater than the diameter of the aperture 304. In other embodiments, the diameter of the upper portion 404 can be less than the diameter of the aperture 304. In further embodiments, the diameter of the upper portion 404 can be equal to the diameter of the aperture 304. The turret 400 can include a grip 406. The grip 406 can include at least one of a contour, a high friction surface, a high friction material, a knurling, and/or one or more grooves, etc., among other possible surfaces and/or materials that include(s) a high or relatively high coefficient of friction, each of which is contemplated herein. The turret 400 can include a depth gauge 408. The depth gauge 408 can include at least one slot 414 and at least one mark 416. The turret can include an aperture 410 that extends the length of the turret (e.g., the turret can be cannulated).

Referring now to FIGS. 13 through 19. In some embodiments, the surgical system 10 can include a rotation arm 500. In various embodiment the rotation arm 500 can include an arc 510. In certain embodiments, the rotation arm 500 can include at least one notch and/or slot 502 placed on and/or within the arc 510. The notch 502 can be configured to accept a k-wire. The rotation arm 500 can include a plurality of notches 502. In some embodiments, the plurality of notches 502 can be spaced evenly along an arc 510. In other embodiments the plurality of notches 502 can be spaced randomly and/or unevenly along the arc 510. In certain embodiments the notches 502 are placed at specific angles on the arc (e.g. at a 10-degree rotation). In various embodiments, the spacing between at least two of the notches 502 approximates a 5-degree (5°) increment. In other embodiments the spacing between at least two of the notches 502 approximates an increment of less than 5 degrees (5°). In alternative embodiments, the spacing between at least two of the notches 502 approximates an increment of greater than 5°.

The rotation arm 500 can include a housing 504. In certain embodiments, the housing 504 can include an aperture 506. In some embodiments, the aperture 506 can be threaded so as to be compatible with the threads of a housing screw 508. In one or more embodiments, the housing screw 508 can be a solid screw (e.g. not cannulated). In various embodiments, the screw 508 can include grips 514 to facilitate rotation of the screw without the use of additional tools. In one or more embodiments the grips 514 are compatible with the use of additional tools. The housing 504 can include a tunnel 512. The tunnel 512 can be configured to conform to at least a portion of handle 334.

The surgical system 10 can be used in a bunion correction surgery, among other procedures that are possible, each of which is contemplated herein.

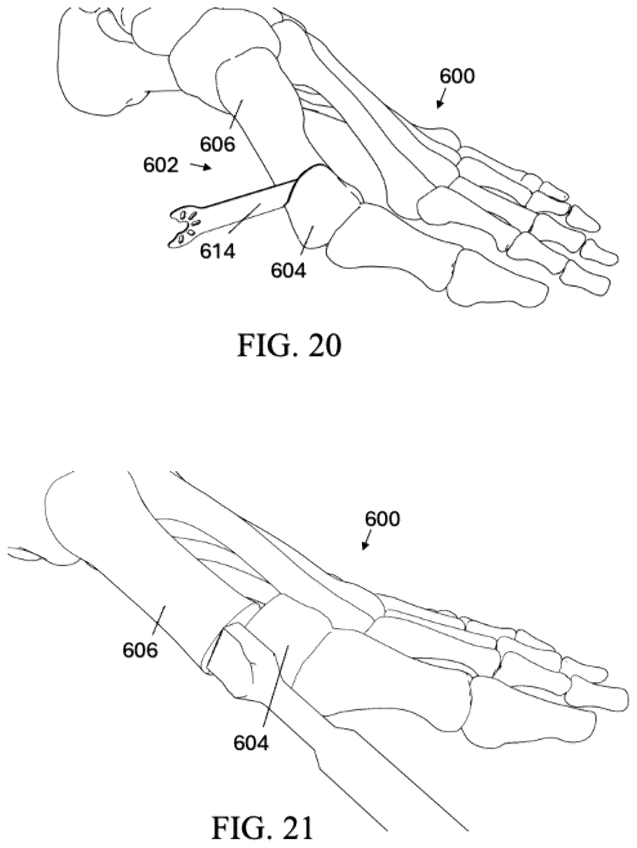

A transverse osteotomy can be performed on the foot 600 of a subject. In some embodiments, the osteotomy is through the first metatarsal 602 (see block 702) (see e.g., FIG. 20). In some embodiments the first metatarsal 602 can be cut with a surgical blade 614. In certain embodiments the osteotomy can separate the first metatarsal 602 into a capital fragment portion 604 and a proximal metatarsal portion 606. The capital fragment 604 can be moved to a desired location (block 704) (see e.g., FIG. 21).

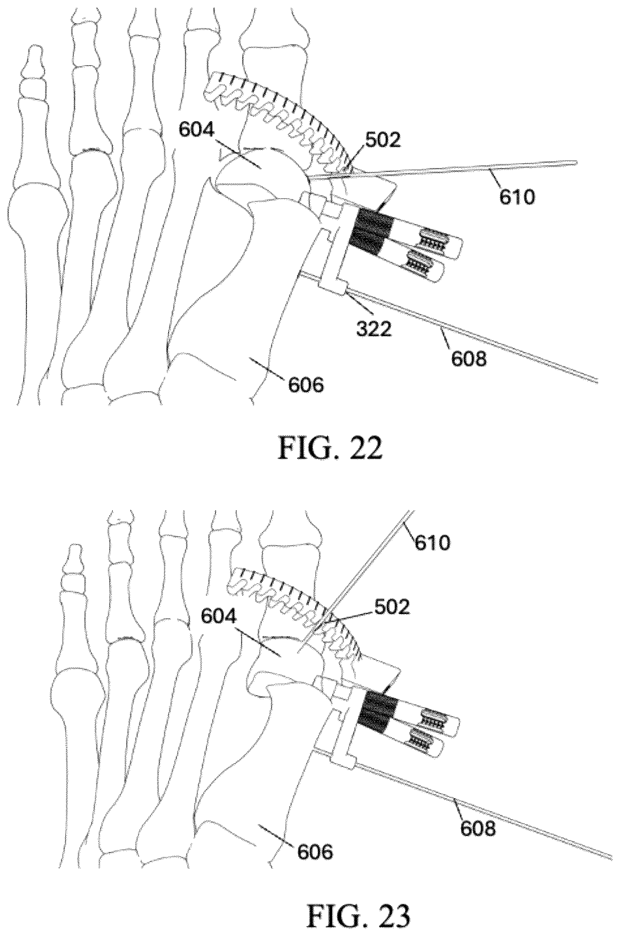

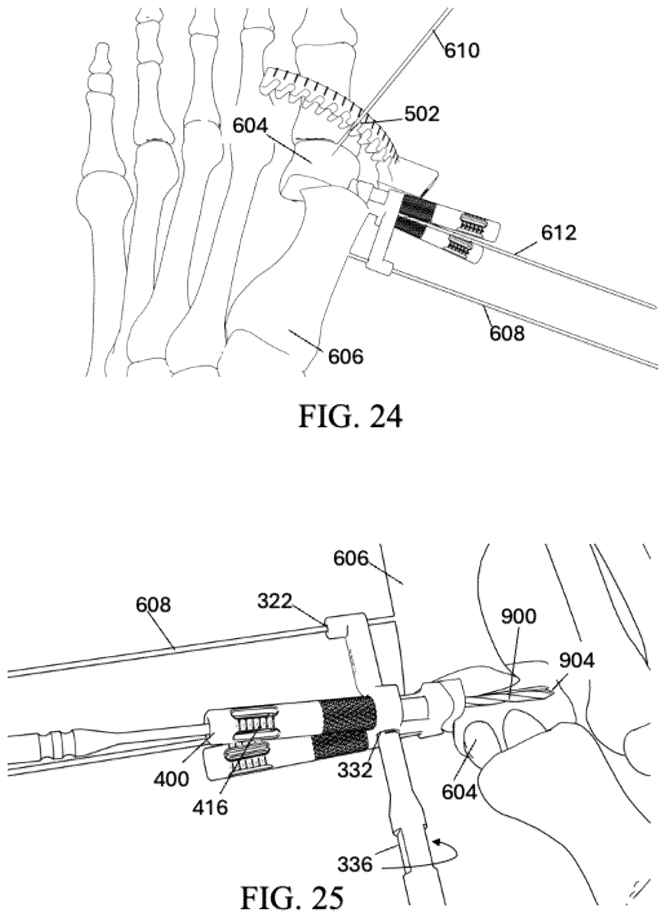

The implant 100 extension 104 can be inserted into the medullary canal of the proximal metatarsal 606 (block 706). A k-wire 608 can be inserted through the aperture 322 and the aperture 106c on the extension 104 and into the proximal metatarsal 606 (block 708) (see e.g., FIG. 22). A k-wire 610 can be inserted through a notch 502 and into the capital fragment 604 (see e.g. FIG. 22). The capital fragment 604 can be rotated by moving the k-wire to another notch 502 (see e.g., FIG. 23). In some embodiments, the rotation and position of the capital fragment 604 can be held by inserting a k-wire 612 through apertures 316 and 126 and into the capital fragment 604 (see e.g., FIG. 24) (block 710).

The drill 900 can be inserted through the turret 400 aperture 410 and into the capital fragment 604. The capital fragment 604 can be drilled until the end 904 of the drill 900 hits the lateral cortex (block 712) (see e.g., FIG. 25). The mark 902 on the drill 900 (see e.g., FIG. 29) can align with a mark 416 on the turret 400. The length of screw needed can be determined from the alignment of the drill mark 902 and the turret mark 416 (block 714). Or the length of screw needed can be determined with a wire gauge. The capital fragment 604 can be drilled through the lateral cortex.

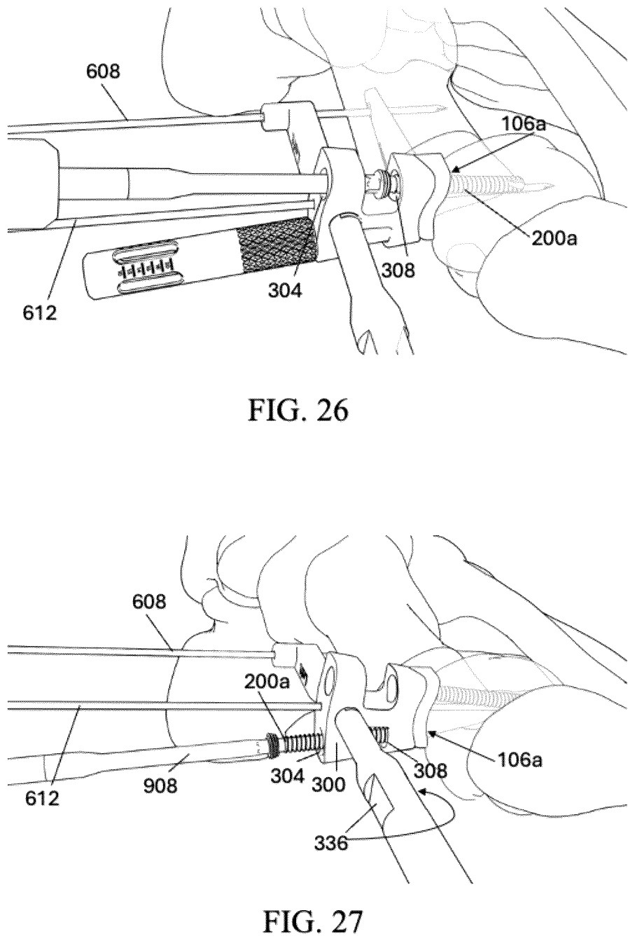

The turret 400 can be removed (block 716) by unscrewing the turret threads 412 from the aperture 106a threads. Bone screw 200a can be inserted through apertures 304, 308, and 106a (block 718) (see e.g., FIG. 26). A screwdriver or other device 908 can be used to rotate the screw 200. In certain embodiments, the screw 200a can be inserted into a single bone portion (e.g., inserted into the capital fragment 604 and not the proximal metatarsal 606). Blocks 712 through 718 can be repeated for additional bone screws (block 720) (see e.g., FIG. 27). K-wire 612 and the guide 300 can be removed (block 722).

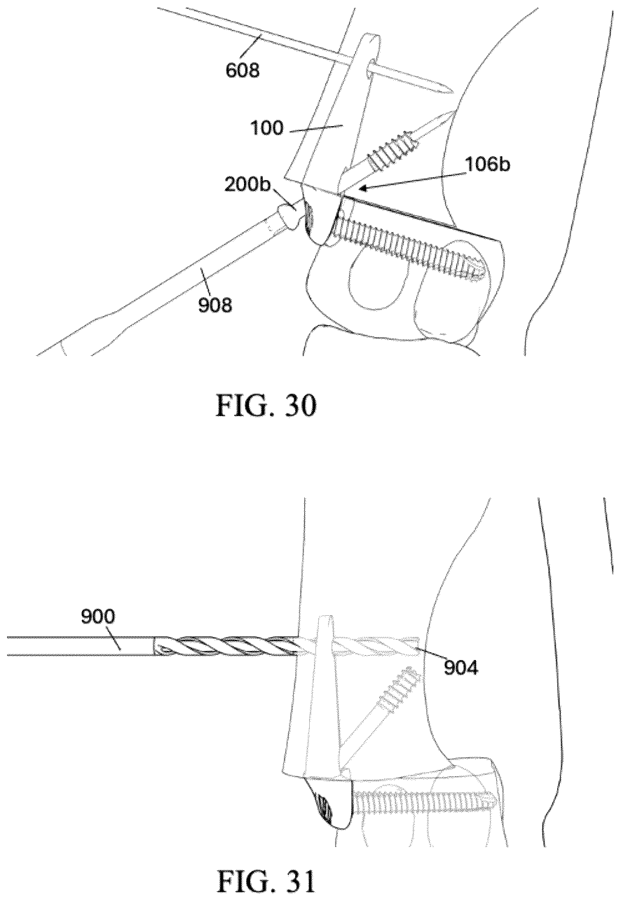

The proximal metatarsal 606 can be drilled until the end 904 of the drill 900 hits the lateral cortex. (block 724). The mark 902 on the drill 900 can align with a mark 416 on the turret 400. The length of screw needed can be determined from the alignment of the drill mark 902 and the turret mark 416 (block 714). Or the length of screw needed can be determined with a wire gauge. The proximal metatarsal 606 can be drilled through the lateral cortex. The turret 400 can be removed and a screw 200b can be inserted into the aperture 106b, compressing the capital fragment 604 to the proximal metatarsal 606 and can be providing rotational stability (see e.g., FIG. 30) (block 726). In certain embodiments, the screw 200b can be inserted into a single bone portion (e.g., inserted into the proximal metatarsal 606 and not into the capital fragment 604). The screw 200b can be at angle angle relative to the axis 108. In some embodiments the screw 200b can be at a sixty-degree (60°) angle. In other embodiments the screw 200b can be at a thirty-degree (30°) angle. In further embodiments the screw 200b can be at an angle greater than sixty degrees (60°). In various embodiments the screw 200b can be at an angle less than sixty degrees (60°).

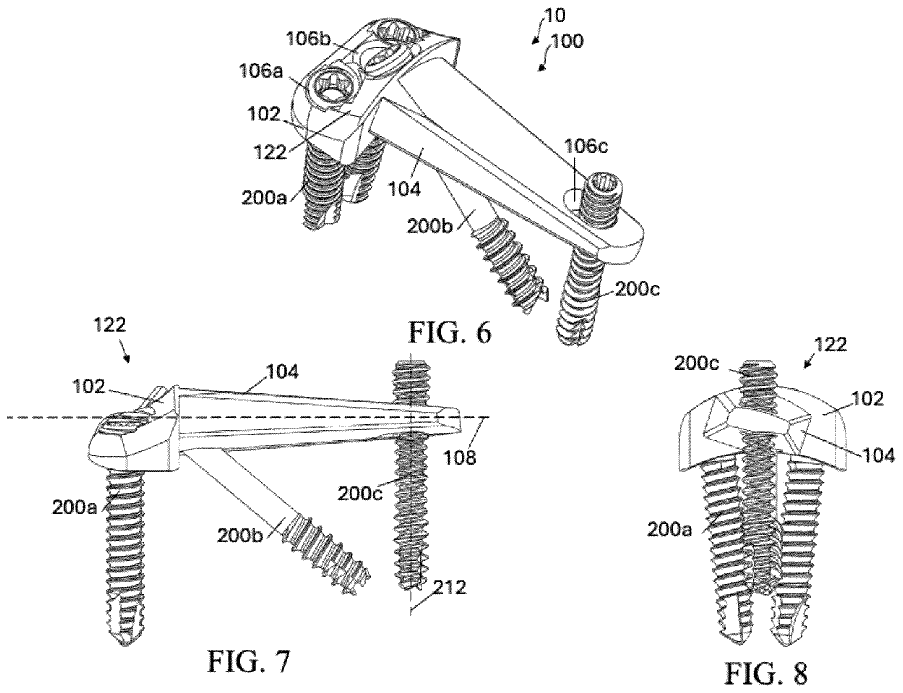

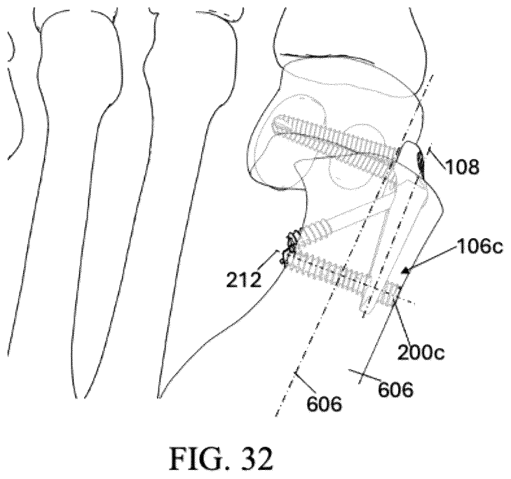

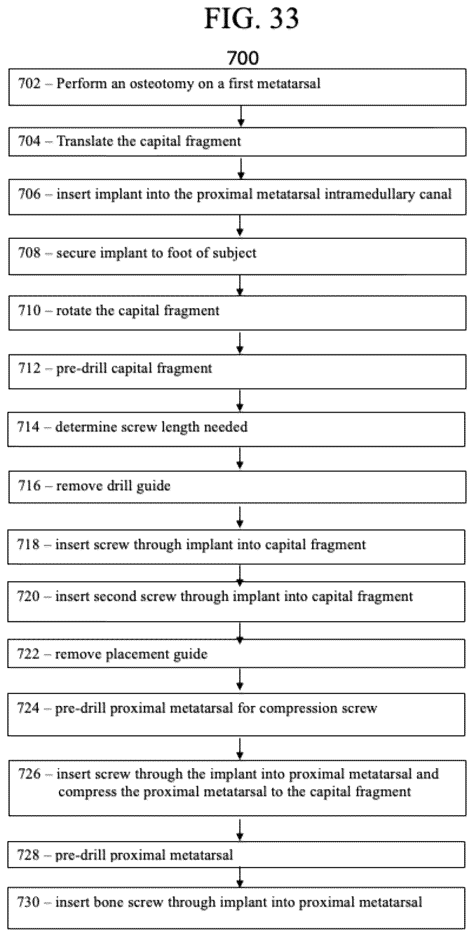

Drill over k-wire 608 (see e.g., FIG. 31) (block 728), insert the bone screw 200c over the k-wire 608 and into the proximal metatarsal 606 (see e.g., FIG. 32) (block 730). Remove k-wire 608 and close the surgical site. In some embodiments the bone screw 200c longitudinal axis 212 can be approximately perpendicular to the extension 104 longitudinal axis 108 (see e.g., FIG. 6) and/or approximately perpendicular to the longitudinal axis 616 of the proximal metatarsal 606. In certain embodiments, the screw 200c can be inserted into a single bone portion (e.g., inserted into the proximal metatarsal 606 and not into the capital fragment 604).

The various embodiments discussed herein may be practiced in other specific forms and the described embodiments are to be considered in all respects only as illustrative, and not restrictive. The scope of the technology is, therefore, indicated by the appended claims rather than by the foregoing description. All changes that come within the meaning and range of equivalency of the claims are to be embraced within their scope. That is, one of ordinary skill in the art will appreciate that modifications and/or adaptations to the various aspects may be made without departing from the scope of the present technology, as set forth in the following claims.

What is claimed is:

1. A surgical apparatus comprising:
an implant comprising:
an attachment body configured to couple the implant to a metatarsal capital fragment, the attachment body comprising one or more first apertures; and
an extension extending from the attachment body and configured for insertion into a metatarsal proximal portion intramedullary canal, the extension comprising one or more second apertures;
a placement guide comprising:
an upper deck including at least one aperture configured to accept a drill guide;
a lower deck including at least one aperture configured to align to the upper deck aperture;
a handle coupled to the guide;
a guide bottom surface configured to conform to a top surface of the attachment body;
a rotation arm removably coupled to the handle, wherein:
the rotation arm includes an arc including one or more notches configured to hold a wire, and
the rotation arm is configured to facilitate rotation of a metatarsal capital fragment and hold the metatarsal capital fragment in a rotated target position, via the wire inserted into the one or more notches;
a housing including a housing aperture; and
a solid housing screw coupled to housing aperture.

2. The surgical apparatus of claim 1, wherein the head of the housing screw includes a plurality of grips to facilitate rotating the housing screw.

3. The surgical apparatus of claim 2, wherein the rotation arm housing further comprises a tunnel configured to conform to at least a portion of the handle.

4. The surgical apparatus of claim 3, wherein the notches are evenly spaced to approximate a 5-degree rotation increment.

5. The surgical apparatus of claim 4, further comprising a first bone screw configured to couple to the implant first aperture.

6. The surgical apparatus of claim 5, further comprising a second bone screw configured to couple to the implant second aperture, wherein the second bone screw is substantially perpendicular to an extension longitudinal axis.

7. The surgical apparatus of claim 6, wherein the second bone screw includes a driver aperture at a first end and includes a self-tapping second end opposite the first end and the second bone screw is fully threaded such that threads extend from the first end to the second end.

8. The surgical apparatus of claim 7, wherein the first bone screw is self-tapping.

9. The surgical apparatus of claim 8, further comprising a third bone screw configured to couple to an implant third aperture, wherein the implant third aperture is configured to guide the third bone screw into the second single bone portion, and the longitudinal axis of the third bone screw is at an acute angle relative to the longitudinal axis of the extension.

10. The surgical apparatus of claim 9, wherein the drill guide comprises a generally cylindrical portion including a depth gauge, wherein the depth gauge includes at least one slot and at least one marking.

11. The surgical apparatus of claim 10, wherein the placement guide further comprises an aperture plug protruding from the guide bottom surface and configured to conform with the implant third aperture.

12. A method comprising:
obtaining a surgical apparatus, the apparatus comprising:
an implant comprising:
an attachment body configured to couple the implant to a metatarsal capital fragment, the attachment body comprising one or more first apertures; and
an extension extending from the attachment body and configured for insertion into a metatarsal proximal portion intramedullary canal, the extension comprising one or more second apertures;
a placement guide comprising:
an upper deck including at least one aperture configured to accept a drill guide;
a lower deck including at least one aperture configured to align to the upper deck aperture;
a handle coupled to the guide;
a guide bottom surface configured to conform to a top surface of the attachment body;
a rotation arm removably coupled to the handle, wherein:
the rotation arm includes an arc including one or more notches configured to hold a wire, and
the rotation arm is configured to facilitate rotation of a metatarsal capital fragment and hold the metatarsal capital fragment in a rotated target position, via the wire inserted into the one or more notches;
a housing including a housing aperture; and
a solid housing screw coupled to housing aperture.

13. The method of claim 12 further comprising:
performing an osteotomy on a metatarsal;
translating the metatarsal capital fragment relative to the metatarsal proximal portion;
inserting a wire through the rotation arm notch into the capital fragment; and
moving the wire to a different notch thereby rotating the capital fragment.

14. The method of claim 13, further comprising:
securing the rotation of the capital fragment by inserting a wire through the guide and the implant;
inserting a drill including a mark through the drill guide and into the capital fragment;
aligning the drill mark with a drill guide mark; and
selecting a screw based on the alignment of the drill mark with the drill guide mark.

15. The method of claim 14, further comprising inserting a first bone screw through the implant first aperture and into a first single bone portion at a fixed angle, wherein the first single bone portion is the metatarsal capital fragment.

16. The method of claim 15, further comprising:
coupling the drill guide to a second implant aperture;
inserting the drill into the drill guide, into the second implant aperture, and into a single bone portion, wherein the single bone portion is the proximal metatarsal portion.

17. The method of claim 16, further comprising:
inserting a second bone screw through the second implant aperture and into a second single bone portion at a fixed angle, wherein the second single bone portion is the proximal metatarsal portion.

18. The method of claim 17, further comprising:
inserting a third bone screw through a third implant aperture and into the second single bone portion, wherein the third implant aperture is a slot.

19. The method of claim 18, wherein the third bone screw is generally perpendicular to the longitudinal axis of the metatarsal proximal portion.

20. The method of claim 19, wherein the third bone screw head is configured to be inserted approximately flush with a surface of the proximal bone portion.
